(19) United States
(12) Patent Application Publication
(10) Pub. No.: US 2017/0209250 A1
(43) Pub. Date: Jul. 27, 2017

(54) VISCOUS GEL DELIVERY METHOD AND APPLICATOR

(71) Applicant: ClearH2O, Inc., Westbrook, ME (US)

(72) Inventors: Jay Palmer, Brunswick, ME (US); Paul Dioli, Yarmouth, ME (US)

(21) Appl. No.: 15/415,213

(22) Filed: Jan. 25, 2017

Related U.S. Application Data
(60) Provisional application No. 62/286,991, filed on Jan. 26, 2016.
(60) Provisional application No. 62/319,822, filed on Apr. 8, 2016.
(60) Provisional application No. 62/381,062, filed on Aug. 30, 2016.

(51) Int. Cl. A61D 7/00; A61M 11/00; B05B 9/04; A01K 45/00

(52) U.S. Cl. CPC A61D 7/00; A01K 45/00; A61M 11/00; B05B 9/0413; A61M 2202/30; A61M 2250/00

(57) ABSTRACT

A method and device for delivering one or more solutions onto a container of animals, such as poultry hatchings, that is being transported along a conveyor line. The method and device are capable of delivering highly viscous gels, having viscosities in the range of 5000 to 10,000 centipoise, and also of delivering multiple solutions to a single container of hatchlings.

### BACKGROUND INFORMATION

Field of the Invention

The invention relates to a method and device for delivering gel beads that are formed from a viscous material for the application of medications, vaccines, nutritive material and other related substances to animals, particularly poultry hatchlings.

Discussion of the Prior Art

Systems for delivering solutions that are used to provide hydration, vaccination, medication, and nutrition to poultry hatchlings are known. The common process is as follows: 1) a large number of hatchlings, typically around 100, are placed in a box that is transported along a conveyor; 2) the box of hatchlings travels under a series of spray cabinets that spray the hatchlings with a number of vaccines and medications that are dispersed in water or, more common recently, a soft flowable gel; and 3) the box of fully vaccinated, medicated, and nutrient-fortified hatchlings is removed from the conveyor and delivered to a poultry raising facility. The water or soft flowable gel is delivered as a spray of small droplets which land on the hatchings and are then consumed by the hatchlings. The spray cabinets commonly use a piston-driven metering pump to deliver the gel from a gel reservoir through the spray cabinets and onto the hatchings. As the hatchings pass under a number of the spray cabinets their downs frequently become wet and matted.

The conventional soft flowable gel usually contains between 90% and 98% water, and includes a setting agent that is typically a kappa or lambda carrageenan, as these carrageenans are known to create a gel that is not very viscous and therefore easily flowable. In general, the amount of setting agent is carefully limited to avoid reducing the flowability of the gel, because less flowable, i.e., more viscous, gels can clog conventional delivery systems and render them inoperable.

More specifically, the conventional gel is formulated to limit the viscosity to a range of 400 to 600 centipoise. For example, the well-known gel Gel-Pac that is manufactured by Animal Science Products is comprised of maltodextrin, hemicellulose extract and dextrans, and is formed by mixing four ounces of the powered ingredients into five gallons of water. This combination is known to produce a gel that has a viscosity between 400 and 600 centipoise depending on the ratio of the dry ingredients.

Viscosity, as presented in this application, is defined using the Brookfield DV1 viscometer with spindle #5 for a five minute test, and with the gel temperature between 68 and 74 degrees Fahrenheit.

On the one hand, increasing the setting agent can cause the applicator to become inoperable, but on the other hand, using too little settling agent can be detrimental to the health of the hatchlings. As mentioned above, the hatchings are repeatedly sprayed with a number of solutions as the container is transported along the conveyor and, with conventional gel formulas, the hatchings downs may become wet and matted. As a result, the hatchings have difficulty consuming the solution. The wet down also causes a loss in body heat, which stresses the hatchlings and may cause sickness, resulting in an increased mortality rate. Consequently, the more viscous the gel, the better it is able to retain its bead form when applied to the hatchlings. As a result, the hatchlings are better able to consume the gel beads, remain dry and better able to thrive.

What is needed, therefore, is a gel delivery process and device thereof that accommodates a gel formulation having a high viscosity.

### BRIEF SUMMARY OF THE INVENTION

The invention is a method of delivering a viscous gel to, for example, poultry hatchlings and other animals having similar vaccination, medication, and/or nutrition needs, and an applicator device for use with the method. Specifically, the method and device are capable of delivering multiple solutions through a single spray cabinet, at least one of the solutions containing a gel having a viscosity in the range of 5,000 to 10,000 centipoises.

The device uses a first pressurized delivery system to deliver a viscous gel through delivery tubes to a plurality of manifolds where the gel is delivered in drop form onto a container of hatchlings traveling along a conveyor. The pressurized delivery system is capable of delivering the gel with greater force than with the conventional piston-based systems, and the plurality of manifolds is capable of providing a relatively even distribution of the viscous gel across and throughout the container of hatchlings.

A second pressurized delivery system is connected to one or more independent nozzles that deliver a second solution to the hatchlings, either to provide two solutions that beneficially interact with one another or to provide two separate solutions, thus eliminating some of the space and machinery that is needed to apply the full range of vaccinations and medications that the hatchlings require.

### BRIEF DESCRIPTION OF THE DRAWINGS

The present invention is described with reference to the accompanying drawings. In the drawings, like reference numbers indicate identical or functionally similar elements. The drawings are not drawn to scale.

FIG. 1 is a front view of the applicator device according to the invention.

FIG. 2 is a front view of the applicator device showing the nozzles.

FIG. 3 is a front view of the device showing the controller.

FIG. 4 is a rear view of the device.

FIG. 5 is a rear view of the device showing the pressure controls and tanks.

FIG. 6 is a front view of the inside of the controller.

FIG. 7 is a top view of a tank.

### DETAILED DESCRIPTION OF THE INVENTION

The present invention will now be described more fully in detail. This invention should not, however, be construed as limited to the embodiments set forth herein; rather, they are provided so that this disclosure will be complete and will fully convey the scope of the invention to those skilled in the art.

The invention is a method and device for delivering vaccinations, medications, nutritive solutions and other similar such substances onto animals, and in particular onto poultry hatchings. The method and device are particularly well suited for creating consumable gel beads from a viscous gel material and delivering the beads onto the hatchlings. The disclosure discusses the invention in terms of its use with poultry hatchlings, or simply “hatchlings”, however, it is understood that it is not limited to use with poultry hatchlings and may be used with other animals.

A gel is provided that has a viscosity that ranges between 5,000 and 10,000 centipoise, which represents a viscosity that resists flow and deformation, that is 10 to 15 times more viscous than the viscosity of gels that are disclosed in the prior art and/or are commercially available. There are a variety of known techniques for increasing the viscosity of a gel. For example, combining a polysaccharide that contains two sulfates per disaccharide with a starch results in a gel having a viscosity up to 10 times that of the soft flowable gels that use other types of sulfated polysaccharides and that are disclosed in the prior art.

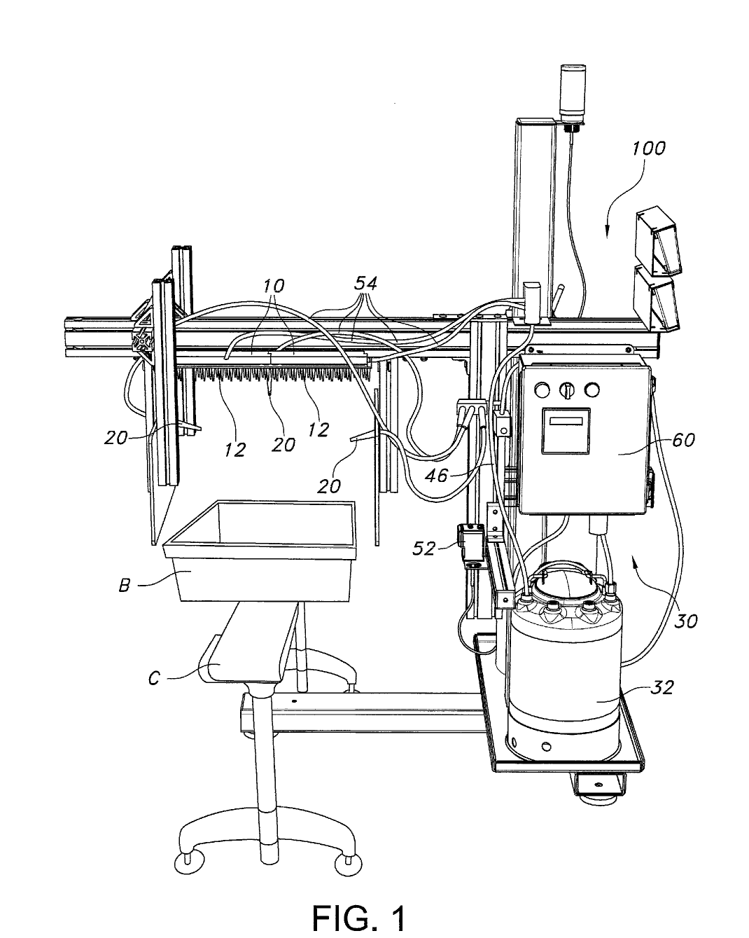

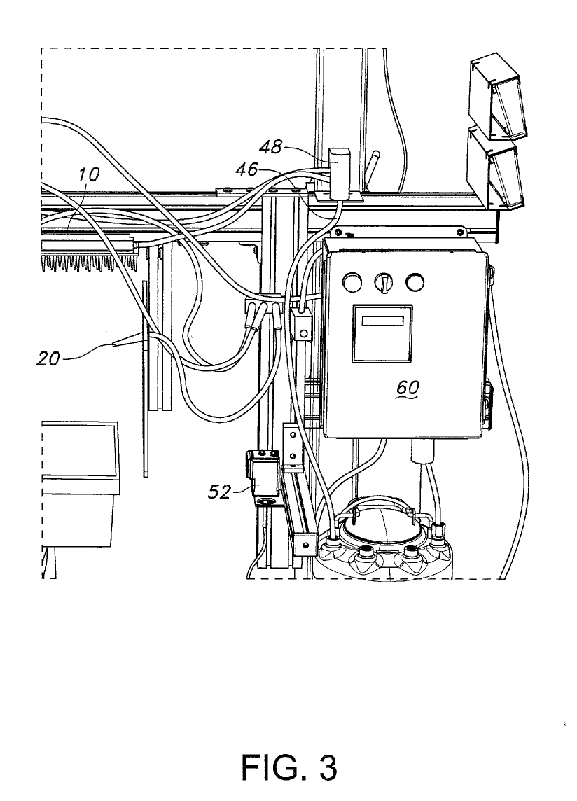

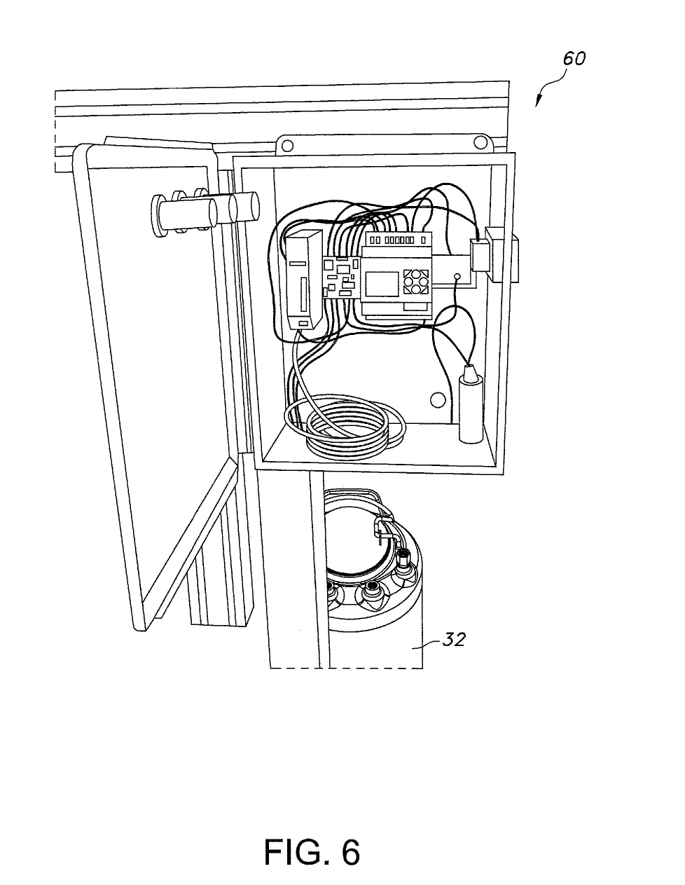

FIGS. 1-7 illustrate a delivery device 100 according to the invention that comprises a plurality of spray manifolds 10, shown in FIGS. 1, 2 and 5, each manifold having a number of dispensing ports 12, one or more independent nozzles 20, a pressurized delivery system 30, shown in FIGS. 1, 4 and 5, and a control unit 60, shown in FIGS. 1, 3 and 6.

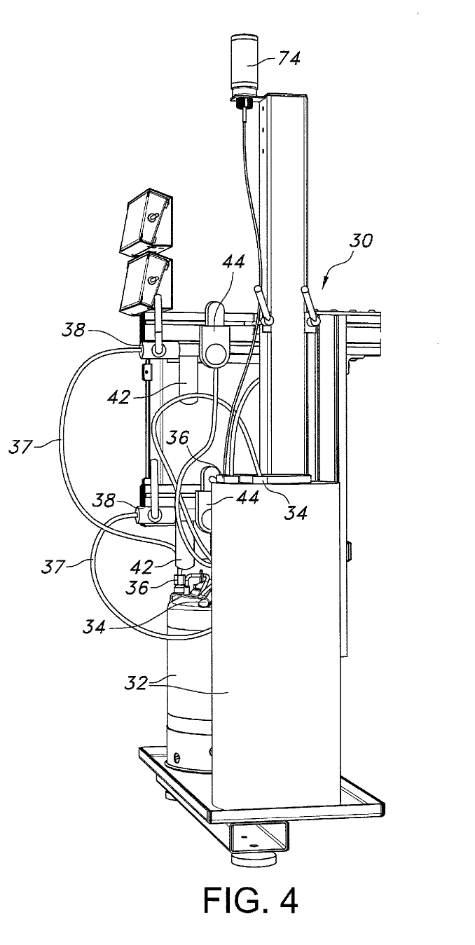

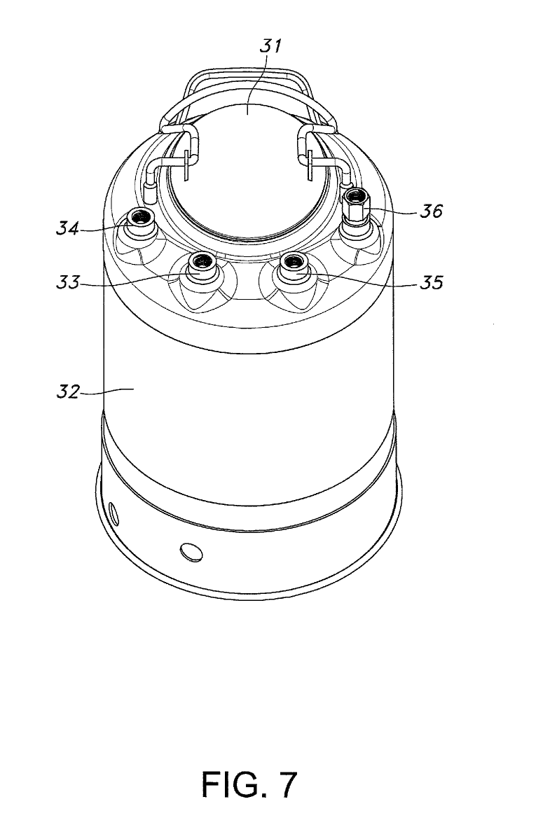

The pressurized delivery system 30 includes at least one pressurized tank 32, shown in FIG. 7, each tank having a lid 31, at least one air intake port 34 and at least one solution exit port 36. FIG. 4 illustrates an air hose 37 that connects on one end to an external compressed air source (not shown) and connects on the other end to an on/off valve 38, which in turn connects to an air filter 42 that connects to a pressure regulator 44. The pressure regulator 44 is connected a controller 60 and to the pressure tank 32 and serves to control the flow of air into the tank 32. The controller 60 is a programmable device, such as a programmable logic controller (PLC), that is programmed to receive input from sensors and perform certain actions, such as opening and closing valves, based on those sensor readings.

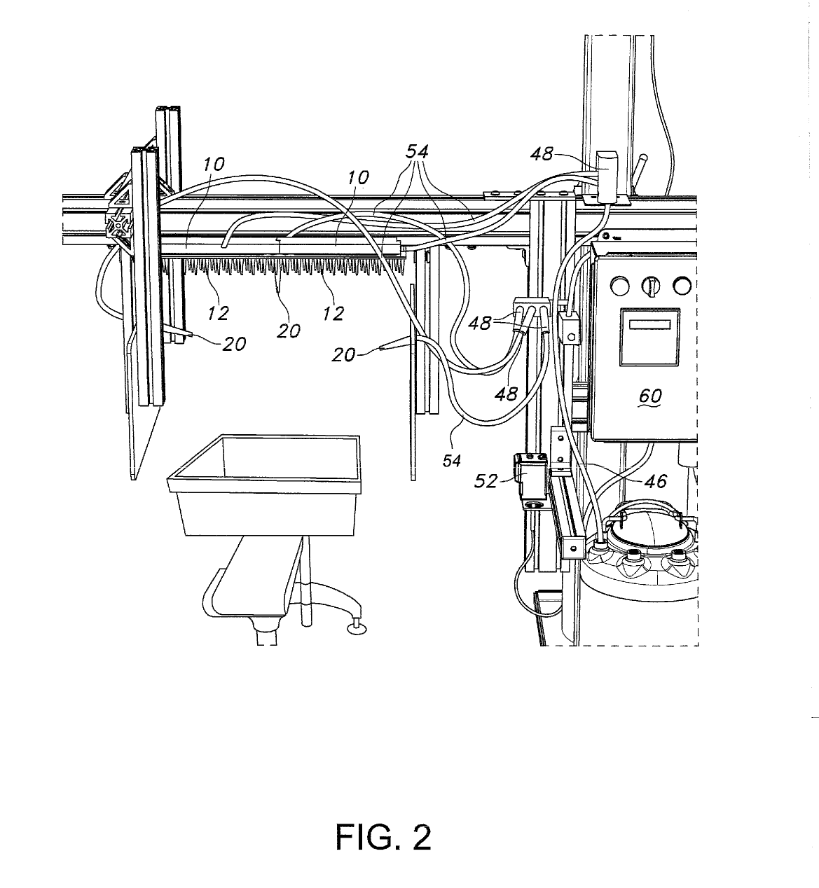

As air is pumped into the pressurized tank 32, the deliverable solution (not shown) is forced out of the tank 32 through a first delivery tube 46 to one or more valves 48. The valves 48 may be any suitable valve, for example, a solenoid valve. One or more secondary delivery tubes 54, shown in FIGS. 2 & 3, connect the valve to the manifolds 10 and/or nozzles 20. In the embodiment shown in FIG. 7 the tank 32 also includes a safety pressure release valve 33 that will release pressure from the tank in the event it exceeds a preset level. A manual pressure release valve 35 is also provided for additional user control.

The use of multiple manifolds is necessary as it is impractical to deliver gels of a high viscosity through a single manifold and obtain even distribution of the viscous gel. For example, the pressure required to provide an appropriate amount of gel through the ports 12 nearest the valve 48 is too weak to deliver an appropriate amount of gel to the ports 12 that are located farthest from the valve 48. Alternatively, providing a pressure that is capable of dispensing the gel from the ports 12 farthest from the delivery tube 54 results in far too much gel emanating through the ports 12 nearest the delivery tube 54. In the embodiment shown, providing pressure in the range of 30-100 PSI is generally sufficient to deliver the viscous gel through the plurality of manifolds 10.

A sensor 52, shown in FIG. 2, is connected to the controller 60 and detects when a box B of hatchlings is about to pass under the manifold 10 and nozzles 20. The sensor 52 may be any suitable sensor that is capable of detecting a container, such as a conventional heat sensor, or a conventional motion sensor such as a standard laser guided sensor. When the sensor 52 notifies the controller 60 of a newly approaching tray, the controller 60 activates the pressurized delivery system 30 for a predetermined period of time. The amount of time that the delivery system 30 delivers the solution is predetermined, based on the size of the containers and the speed of the conveyor.

The embodiment shown includes two pressurized delivery systems 30 for delivering two solutions. In the first system 30 one valve 48 has two secondary delivery tubes 54 for delivery of the solution from the valve to a pair of manifolds 10. In the second system 30, three valves 48 are connected to three secondary delivery tubes 54 for delivering the solution to three independent nozzles 20.

The combination of spray manifolds 10 and independent nozzles 20 allows an operator to dispense one or more solutions in a manner that provides greater coverage of the chicks and/or that allows for the delivery of multiple solutions in a single spray cabinet. For example, it may be desirable to provide two solutions that interact with one another by using the manifolds 10 to deliver a first substance and using the independent nozzles 20 to deliver a second substance, either simultaneously or sequentially. Alternatively, it may be desirable to use a single spray cabinet to deliver multiple substances simply to save time and space.

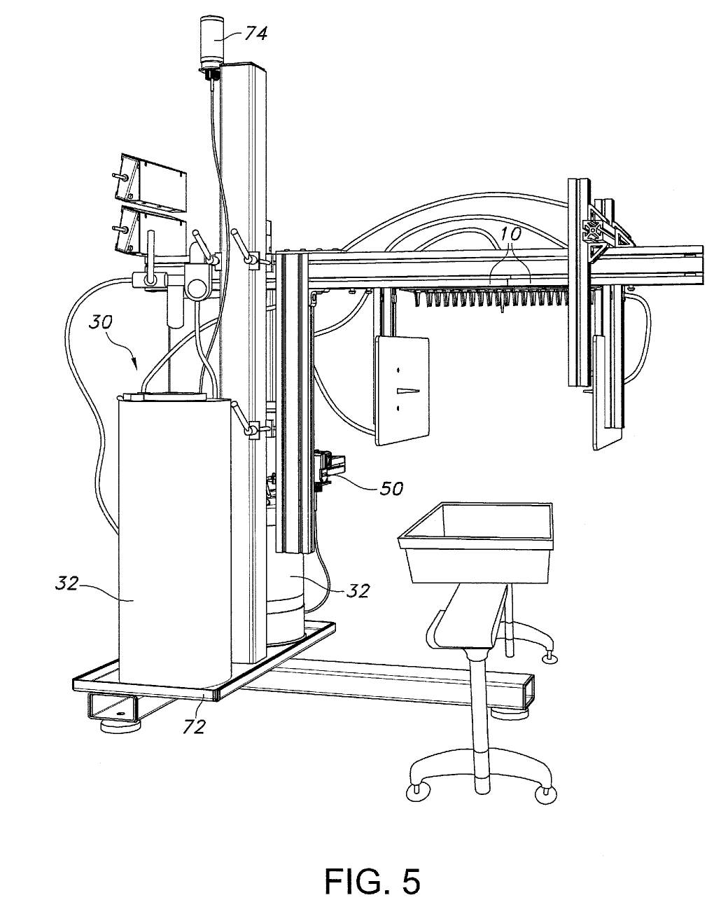

A scale 72, illustrated in FIG. 5, and an alarm system 74 that is integrated into the control unit 60 are provided to alert operators when the gel tank 32 is approaching a low level of gel. The scale 72 is affixed to the bottom of the applicator 100 and beneath the tank 32 and is connected to the alarm system 74 in the control unit 60 by any suitable means. When the weight of the tank 10 reaches a certain level the scale 72 will transmit that information to the alarm system and an alarm will signal the need to change tanks 32.

A group of hatchlings, typically around 100, are placed in an open top container B and transported along a conveyor C towards the applicator device 100. The sensor 52 detects the container as it approaches the applicator device 100 and opens the valve 48, allowing the solution in the pressurized tank 32 to flow through the secondary delivery tubes 54, to be dispensed in an approximately even distribution as droplets or beads onto the hatchings. When the programmed amount of time has passed, the controller 60 sends a signal to close the valve 48, thereby shutting off the supply of pressurized solution.

It is understood that the embodiments described herein are merely illustrative of the present invention. Variations in the viscous gel delivery method and device may be contemplated by one skilled in the art without limiting the intended scope of the invention herein disclosed and as defined by the following claims.

What is claimed is:

1: A method of delivering gels to hatchlings that are transported in a container along a conveyor, the method comprising the steps of:
a) providing a gel having a viscosity of between 5,000 and 10,000 centipoise;
b) providing an applicator device, the device comprising a pressurized tank for storing the gel that is connected to a delivery tube that extends to a valve, the valve being connected to a plurality of delivery tubes that are connected to a corresponding number of manifolds that include a plurality of dispensing openings, and a sensor that detects the container of hatchings as it moves beneath the manifolds and opens the valve for as long as the container is passing beneath the manifolds; and
c) dispensing the gel from the applicator device in the form of gel bead droplets onto the hatchings as the container passes beneath the manifold.

2: A device for delivering one or more solutions onto hatchings, the device comprising:
a plurality of spray manifolds, each manifold having a number of dispensing ports for dispensing a solution;
a first pressurized delivery system having a first solution tank containing a first solution; and
a control unit;
wherein the first pressurized delivery system delivers the first solution from the solution tank to the spray manifolds; and
wherein the control unit controls how much of the first solution is dispensed through the manifolds onto the hatchlings.

3: The device of claim 2 wherein the first solution is a gel having viscosity between 5000 and 10,000 centipoise.

4: The device of claim 2 further comprising a second pressurized delivery system having a second solution tank containing a second solution and one or more independent nozzles; and
wherein the control unit is programmed to deliver the first solution and the second solution as the container of hatchlings pass under the plurality of manifolds and the independent nozzles.
